(19) United States
(12) Patent Application Publication
(10) Pub. No.: US 2016/0011191 A1
(43) Pub. Date: Jan. 14, 2016

(54) DETECTION KIT AND DETECTION METHOD

(71) Applicant: National Tsing Hua University, Hsinchu City (TW)

(72) Inventors: Chung-Yao YANG, Hsinchu City (TW); Chao-Kai HSU, Hsinchu City (TW); Chao-Min CHENG, Hsinchu City (TW)

(21) Appl. No.: 14/558,502

(22) Filed: Dec. 2, 2014

(30) Foreign Application Priority Data
Jul. 11, 2014 (TW) 103124016

(51) Int. Cl. G01N 33/564; G01N 1/12; G01N 33/543

(52) U.S. Cl. CPC G01N 33/564; G01N 33/54386; G01N 1/12; G01N 2001/028

(57) ABSTRACT

A detection kit applied for detecting a body fluid sample of an organism includes a sampling device and a detecting device. The sampling device includes a sampling unit and a filtering unit detachably covering the sampling unit. The sampling unit collects the body fluid sample that is filtered by the filtering unit. The detecting device includes at least one reaction zone including a fiber substrate and a detecting reagent immobilized to the fiber substrate. The reaction zone is disposed as the fiber substrate contacts the body fluid sample collected by the sampling unit. The fiber substrate absorbs the body fluid sample collected by the sampling unit, so that the body fluid sample reacts with the detecting reagent for the detection. A detection method for detecting a body fluid sample is also disclosed. This invention is advantageous for simplified pretreatment, less amount of the reagent and rapid analysis.

### CROSS REFERENCE TO RELATED APPLICATIONS

This Non-provisional application claims priority under 35 U.S.C. §119(a) on Patent Application No(s). 103124016 filed in Taiwan, Republic of China on Jul. 11, 2014, the entire contents of which are hereby incorporated by reference.

### BACKGROUND OF THE INVENTION

1. Field of Invention

The invention relates to a detection kit and a detection method and, in particular, to a detection kit and detection method applied to a biomedical detection.

2. Related Art

Clinically, the instrument for collecting a specimen (sample) of an organism is mainly divided into a brush-head type, a film type and a curette type. When the specimen of the endometrial cancer is detected, a sample collector is placed on the organism to be collected, and the endometrium cell and secretion thereof can be obtained by absorbing or curetting the endometrium surface and then treated with the processing and detection. There are further many detections clinically where the sample collector is used to collect the body fluid of the affected part of an organism for the judgment of the disease process or treatment effect. However, the sample collected by the conventional sample collector always carries some unnecessary objects. For example, the endometrium cell and the secretion thereof are usually mixed together. Therefore, if the detection needs to be performed only to the secretion, the cell and the secretion will be separated from each other, so the pretreatment before the detection of the secretion will show some complexity.

Furthermore, in the clinical application, after the sample collection is finished, the collected sample or specimen needs to be transferred to the detecting apparatus or analyzing apparatus to undergo, for example, the ELISA (enzyme-linked immunoadsorbent assay) detection. However, in the ELISA detection conventionally performed by a 96-well plate, the amount of the analyzed sample and reagent requires 50˜100 microlitres each time. Besides, the reagent and sample need to be reacted at a particular temperature (such as 37° C.), and the reaction and washing steps need to be performed repeatedly and each of the steps takes about 10 minutes to 1 hour, and finally, the spectrometer is required to detect the light-absorbing value to obtain the analysis result. The all steps are complicated and take a lot of time. Although the required amount of the sampling and reagent can be reduced by using the Dot-ELISA (also called Dot-Blot) detection method, the reaction and washing steps which take a large amount of time are still unavoidable.

Therefore, it is an important subject to provide a detection kit and a detection method which can reduce the required amount of the sample and have advantages such as simplifying the pretreatment before the detection process and speeding up the process so as to enhance the clinical applicability.

### SUMMARY OF THE INVENTION

In view of the foregoing subject, an objective of the invention is to provide a detection kit and a detection method which can reduce the required amount of the sample and have advantages such as simplifying the pretreatment before the detection and speeding up the process so as to enhance the clinical applicability.

To achieve the above objective, a detection kit according to the invention is applied for detecting a body fluid sample of an organism and comprises a sampling device and a detecting device. The sampling device includes a sampling unit and a filtering unit detachably covering the sampling unit. The sampling unit collects the body fluid sample that is filtered by the filtering unit. The detecting device includes at least one reaction zone including a fiber substrate and a detecting reagent immobilized to the fiber substrate. The reaction zone is disposed as the fiber substrate contacts the body fluid sample collected by the sampling unit, and the fiber substrate absorbs the body fluid sample collected by the sampling unit, and the body fluid sample reacts with the detecting reagent for the detection.

In one embodiment, the detecting device includes at least one non-reaction zone which is covered with hydrophobic material.

In one embodiment, the non-reaction zone surrounds the reaction zone to expose the fiber substrate to absorb the body fluid sample collected by the sampling unit.

In one embodiment, the detection kit is for the bullous pemphigoid (BP).

In one embodiment, the detecting reagent includes a plurality of COL17 antigens.

In one embodiment, the material of the filtering unit includes gauze, nylon cloth, non-woven cloth or their any combination.

To achieve the above objective, a detection method according to the invention for detecting a body fluid sample of an organism comprises steps of: providing a sampling device and a detecting device, wherein the sampling device includes a sampling unit and a filtering unit detachably covering the sampling unit, the detecting device has at least one reaction zone which includes a fiber substrate and a detecting reagent immobilized to the fiber substrate; making the sampling unit covered by the filtering unit contact the body fluid sample so that the body fluid sample is absorbed by the sampling device; separating the filtering unit from the sampling unit and making the body fluid sample absorbed by the sampling unit contact the reaction zone; and detecting the body fluid sample.

In one embodiment, the detection method is for the BP.

In one embodiment, the detecting device includes a plurality of COL17 antigens immobilized to the fiber substrate.

In one embodiment, the detection method further comprises a step of: detecting the interaction between the body fluid sample and the COL17 antigens.

As mentioned above, in the detection kit and detection method of the invention, the sampling device and the detecting device can effectively implement the collection and detection to the body fluid sample. The sampling unit of the sampling device is covered with a filtering unit, which can effectively separate non detection target from the body fluid sample so that the sampling unit can effectively collect the desired target from the body fluid sample. Thereby, the pretreatment and the pretreatment time for the body fluid sample can be lessened and the problem of losing body fluid sample due to the extra processing steps can be avoided. Therefore, the required amount of the sample can be reduced. Besides, the detection kit of the invention includes a detecting device, so the detection purpose can be achieved by making the body fluid sample that is filtered and absorbed by the sampling unit contact the detecting device.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a schematic flowchart of a detection method of an embodiment of the invention;

FIG. 2 is a schematic assembled diagram of a detection kit of an embodiment of the invention;

FIG. 3 is a schematic exploded diagram of the detection kit in FIG. 2;

FIG. 4A is a schematic diagram showing the step S13 of the detection method in FIG. 1;

FIG. 4B is a schematic diagram showing the step S15 of the detection method in FIG. 1;

FIG. 5A is a schematic top view of the detecting device of the detection kit in FIG. 1;

FIG. 5B is a schematic enlarged diagram of a part of the region A of the detecting device in FIG. 5A;

FIG. 5C is a schematic sectional diagram taken along the line A-A in FIG. 5A;

FIG. 6A is a schematic diagram of a detecting device of another embodiment of the invention;

FIG. 6B is a schematic sectional diagram taken along the line a-a in FIG. 6A;

FIG. 7 is a schematic diagram of a detecting device of another embodiment of the invention; and

FIG. 8 is a schematic diagram showing the relative intensity result of the anti-NC16A antibody obtained form the body fluid of the body surface of the patient by using a paper-based detecting device applied to the ELISA.

### DETAILED DESCRIPTION OF THE INVENTION

The present invention will be apparent from the following detailed description, which proceeds with reference to the accompanying drawings, wherein the same references relate to the same elements.

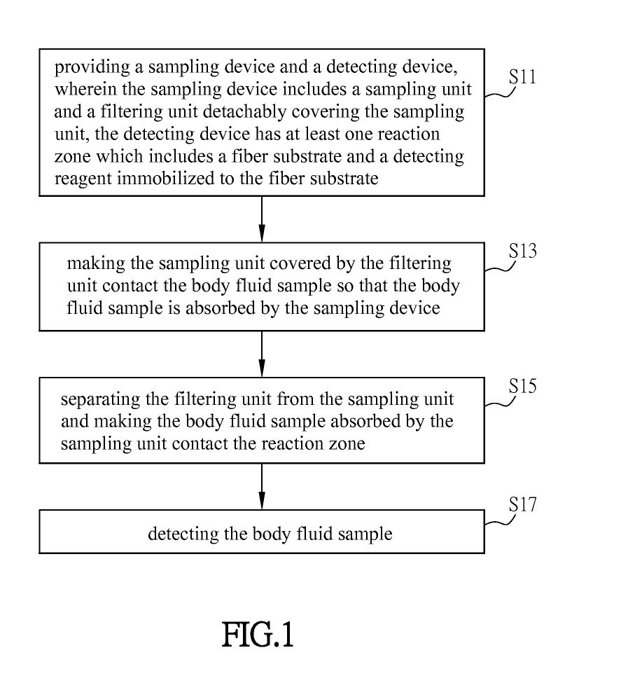

FIG. 1 is a schematic flowchart of a detection method of an embodiment of the invention. As shown in FIG. 1, in this embodiment, the detection method is used to detect a body fluid sample of an organism, including the steps of: providing a sampling device and a detecting device, wherein the sampling device includes a sampling unit and a filtering unit detachably covering the sampling unit, the detecting device has at least one reaction zone which includes a fiber substrate and a detecting reagent fixed to the fiber substrate (step S11); making the sampling unit covered by the filtering unit contact the body fluid sample so that the body fluid sample is absorbed by the sampling device (step S13); separating the filtering unit from the sampling unit and making the body fluid sample absorbed by the sampling unit contact the reaction zone (step S15); and detecting the body fluid sample(step S17).

For making the related detail of the detection method clearer, the detection kit with the included elements and operations will be illustrated as below, and then how to implement the detection method by the detection kit will be illustrated. To be noted, the following embodiments are just for the illustrative purpose but not for limiting the scope of the invention.

To be noted, the detection kit and detection method of this invention are suitable for the disease detection, the judgment of the disease process, the treatment process, or the physiological condition estimation of an organism. The object to be detected in this embodiment is obtained from the body fluid sample of an organism, such as the body fluid generated by the non-traumatic tissue, which is, for example, the body fluid secreted by the endometrium or oral mucosa, and favorably the body fluid sample of the body surface of an organism. The so-called body fluid of body surface can be saliva, plasma, lymph or tissue fluid for example.

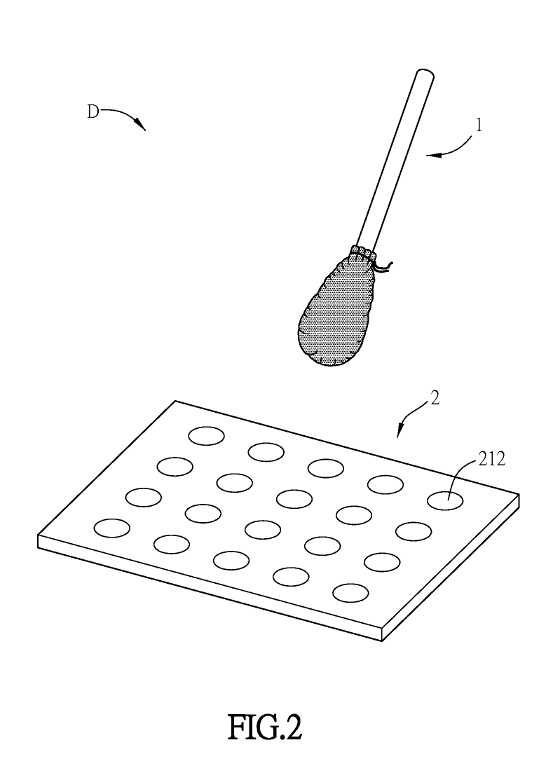

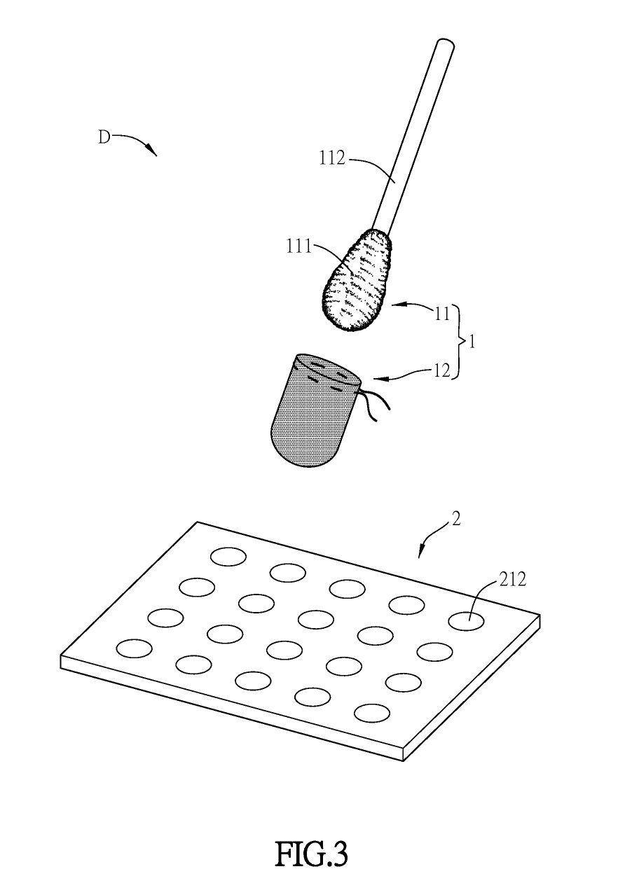

FIG. 2 is a schematic assembled diagram of a detection kit of an embodiment of the invention, and FIG. 3 is a schematic exploded diagram of the detection kit in FIG. 2. As shown in FIGS. 1, 2, 3, in the step S11 of the detection method of this embodiment, a sampling device and a detecting device are provided. In detail, the detection kit D includes a sampling device 1 and a detecting device 2. The sampling device 1 is used to collect the body fluid sample of the organism, and the detecting device 2 is used to detect the body fluid sample collected by the sampling device 1. The structural features of the above-mentioned elements are illustrated as below.

In this embodiment, the sampling device 1 includes a sampling unit 11 and a filtering unit 12, and the filtering unit 12 detachably covers the sampling unit 11. Herein as an example, the sampling unit 11 includes a sampling portion 111 (such as cotton material) and a holding portion 112 (such as a bar). In actual applications, the sampling portion 111 can be any substance that is capable of collecting the body fluid sample of the organism and is, for example but not limited to, cotton, foam rubber, or other materials with absorptiveness, or their any combination.

The filtering unit 12 is a filter net structure. When the filter unit 12 covers the sampling unit 11 for the collection of the body fluid sample, the meshes of the filtering unit 12 can filter out the undesired part, i.e. not the detection target, from the body fluid sample. To be noted, the filtering in this embodiment indicates that the designated particulate matters, particles, or other small-sized solid substances in the flowing fluid are stopped or intercepted on one side of the meshes so as to be separated from the fluid portion flowing to the other side of the meshes. Moreover, the filtering in this embodiment also can allow some error which may be caused due to the process defect or a few special condition or tolerated theoretically or empirically, and thus may result in an incomplete separation.

The filtering unit 12 of this embodiment is, for example but not limited to, gauze, nylon cloth, non-woven cloth or their any combination. In detail, by taking the body fluid of a wound on the body surface of an organism or the secretion on the female endometrium as an example, the filtering unit 12 can filter out the non detection targets (e.g. the injured cell of the wound or mucous membrane tissue) from the body fluid, so as to prevent the non detection targets like injured cell of the wound or mucous membrane tissue from adhering to the sampling unit 11 covered by the filtering unit 12. The sizes of the filtering unit 12 and meshes are not limited in this invention. The size of the filtering unit 12 can be designed according to the sampling unit 11 covered by the filtering unit 12, and the filtering unit 12 can be selected according to the ingredients of the body fluid of the body surface and the detection requirement and can be adjusted according to the actual situation and engineering design.

The filtering unit 12 of this embodiment is shaped like a covering bag. In actual applications, a binder, or a fixing element that can be rapidly dismounted, or the like can be disposed around the bag hole of the filtering unit 12, so that the filtering unit 12 is fixed to the sampling unit 11 and is prevented from being falling off during the collection process.

The outer surface of the filtering unit 12 relative to the sampling unit 11 can be coated with a lubricant, which is, for example but not limited to, petrolatum, so that the user to be detected (i.e. a subject) can feel more comfortable when detected by the sampling device 1.

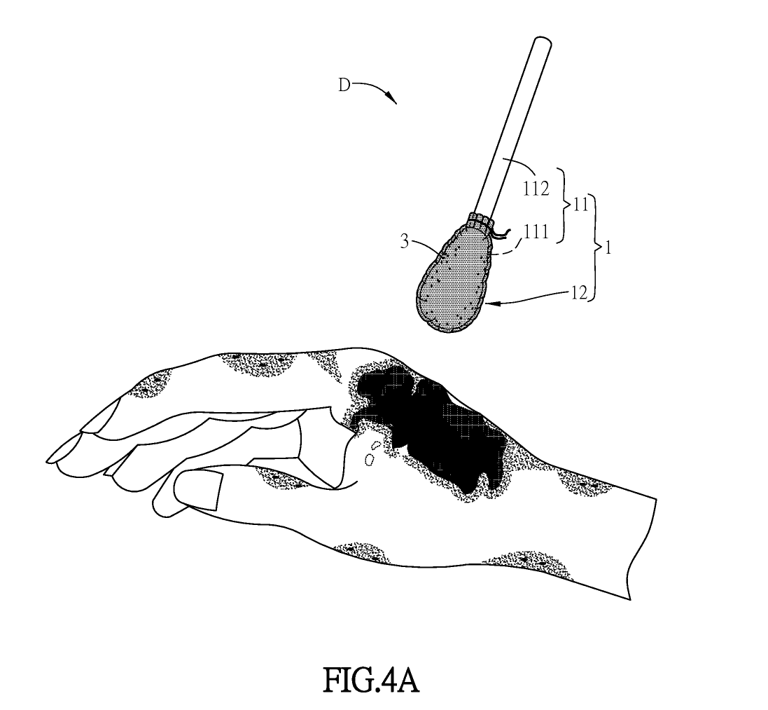

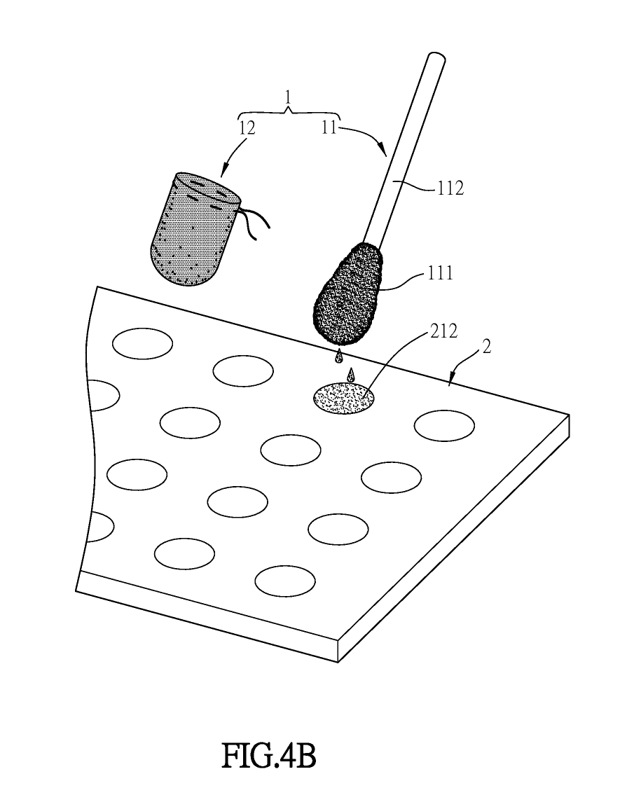

FIG. 4A is a schematic diagram showing the step S13 of the detection method in FIG. 1. As shown in FIGS. 1-4A, according to the above-mentioned structure of the sampling device 1, in the step S13, the sampling unit 11 covered by the filtering unit 12 contacts the body fluid sample so that the body fluid sample can be absorbed by the sampling device 1. In detail, the user can hold the holing portion 112 and make the sampling portion 111 covered by the filtering unit 12 contact the body fluid sample of the subject and contact, for example, the blister of the subject to absorb the secreted body fluid. Although the sampling portion 111 contacting the blister of the subject is illustrated as an example, the sampling unit 11 of this embodiment also can be used to collect the body fluid sample of the endometrium or oral mucosa. When the sampling device 1 collects the body fluid sample, the filtering unit 12 is usually stained with some substances not belonging to the detection target, such as cuticle tissue, mucous membrane tissue or other non detection targets 3, and these non detection targets 3 may be undesired for the disease detection or physiological condition detection or even affect the detection result. Since the size of the meshes of the filtering unit 12 is less than that of the non detection target 3, the non detection target 3 can't pass through the filtering unit 12 or contact or adhere to the sampling portion 111.

FIG. 4B is a schematic diagram showing the step S15 of the detection method in FIG. 1. As shown in FIGS. 1-4B, according to the above-mentioned detection kit D, in the step S15, after collecting the body fluid sample, the filtering unit 12 and the sampling unit 11 are separated from each other and the sampling unit 11 absorbing at least a portion of the body fluid sample is made contact the reaction zone 212 so that the body fluid sample can adhere to the reaction zone 212 of the detecting device 2. In detail, some methods of making the body fluid sample absorbed by the sampling unit 111 adhere to the reaction zone 212 are illustrated, for example but not limited to, as below. For example, the sampling portion 111 can be directly laid on the reaction zone 212, or the sampling portion 111 can be eluted and then the body fluid sample absorbed thereon can drop to the reaction zone 212, or the sampling portion 111 is directly squeezed by a tool so that the body fluid sample absorbed thereon can drop to the reaction zone 212. However, the above methods are not for limiting the scope of the invention and can be designed according to the type of the body fluid sample or the detection requirement.

After the step S15, the step S17 is to detect the body fluid sample disposed in the reaction zone 212. In this embodiment, the detecting device 2 of the detection kit D is used for the detection, and the operation details of the detecting device 2 are illustrated as below.

To be noted, although the body fluid sample to be detected is not limited to a particular type, the bullous pemphigoid (BP) is illustrated as an example here for the clear description. That is, the body fluid sample to be detected includes, for example, an antibody that is generated by the BP patient anti COL17 antigen (or called BP180 and BPAG2), such as blood serum, and the body fluid sample is favorably the body fluid generated by the blister of the BP patient.

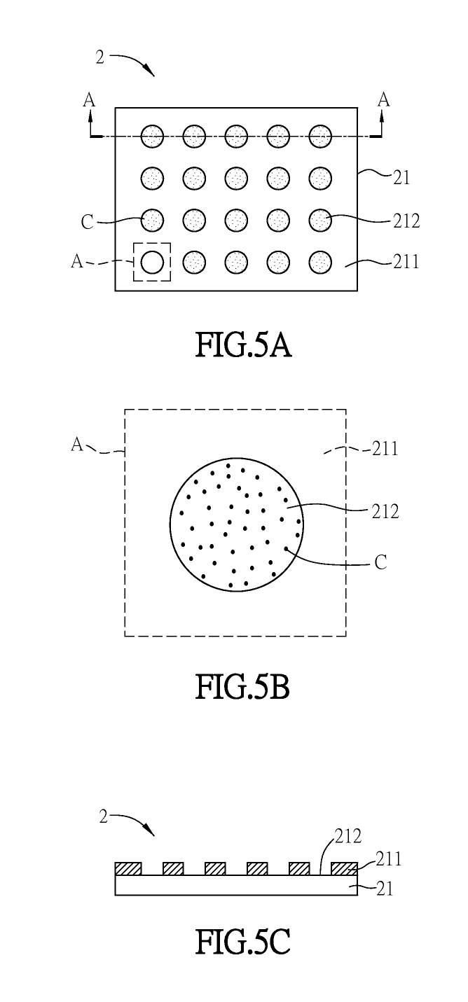

FIG. 5A is a schematic top view of the detecting device of the detection kit in FIG. 1, FIG. 5B is a schematic enlarged diagram of a part of the region A of the detecting device in FIG. 5A, and FIG. 5C is a schematic sectional diagram taken along the line A-A in FIG. 5A. As shown in FIGS. 5A to 5C, in this embodiment, the detecting device 2 is a substrate 21, which includes at least a non-reaction zone 211 and at least a reaction zone 212. Herein as an example, the substrate 21 has a non-reaction zone 211, and a plurality of reaction zones 212 can be defined by the disposition of the non-reaction zone 211. In this embodiment, the reaction zones 212 are separated by the non-reaction zone 211. To be noted, the number, shape and size of the reaction zone shown here are not for limiting the scope of the invention, and they can be designed according to the experimental condition.

As shown in FIGS. 5A and 5B, in this embodiment, the non-reaction zone 211 of the substrate 21 is covered with a hydrophobic material so it also can be called a hydrophobic zone. In detail, the non-reaction zone 211 of this embodiment is made by the wax printing method. In the wax printing method of forming the hydrophobic non-reaction zone 211, a device with wax jet function, such as a printer, is used to dispose the wax on the substrate 21 according to the pattern, shape or size specified by the user. By the disposition of the hydrophobic non-reaction zone 211, the body fluid of the body surface can be effectively restricted within the reaction zones 212 so that the sample can be prevented from being lost and the detection accuracy can be enhanced.

The non-reaction zone 211 can be made not only by the above-mentioned method but also by other methods. For example, the substrate 21 with hydrophobicity can be coated with a photoresist layer. As an embodiment, when the SU-8 epoxy-based negative photoresist is applied, the region illuminated by the UV light will not dissolve in the developer solution so the hydrophobic non-reaction zone 211 can be formed thereby, whereas the region not illuminated by the UV light can form the hydrophilic reaction zone 212. This kind of the method can be comprehended by those skilled in the art and therefore is not described here for conciseness.

The substrate 21 can be made by fiber material, e.g. the vegetative fiber, and favorably by the high-density fiber. In this embodiment, the average aperture of the high-density fiber substrate is 0.7 μm˜12 μm and favorably 1 μm˜10 μm. The average aperture of the high-density fiber substrate also can have a narrower range within the above-mentioned ranges. When the aperture of the substrate is larger, the body fluid sample flowing thereon can be affected by the siphon and capillarity forces so as to have a larger speed, whereas when the aperture of the substrate is smaller, the body fluid sample flowing thereon is mainly affected by the siphon force so as to have a smaller speed. In view of the detection result, the larger flowing speed is unfavorable for the detection target immobilized in the reaction zone 212, whereas the smaller flowing speed may result in more non-specific bindings. Therefore, in actual applications, the magnitude of the average aperture of the high-density fiber substrate can be determined according to the ingredients of the body fluid sample and the detection requirement and can be adjusted according to the actual condition and engineering design.

In this embodiment, since the reaction zones 212 are defined and surrounded by the hydrophobic non-reaction zone 211 (i.e. a partial region of the substrate 21), the reaction zones 212 also have the hydrophilicity of the fiber substrate. The reaction zone 212 retains and absorbs the body fluid of the body surface mainly by the capillary effect generated in the fiber substrate. Furthermore, the body fluid of the body surface can be affected by the wick absorbing, diffusion and transmission effects in the reaction zone 212 due to the density of the fiber substrate and the minute groove in the fiber substrate. In comparison with the conventional case where nitrocellulose is used as the material absorbing the sample to be detected so it only can absorb the sample to be detected on the surface, the high-density fiber substrate applied in this embodiment has better water permeability and the reaction zones 212 are surrounded by the hydrophobic non-reaction zone 211 formed by the wax printing, so that the fiber substrate can be exposed to absorb the body fluid sample that is collected by the sampling unit and the body fluid sample can be restricted within the reaction zones 212. Hence, the body fluid sample can be retained more effectively, so the detection accuracy of the following ELISA can be increased.

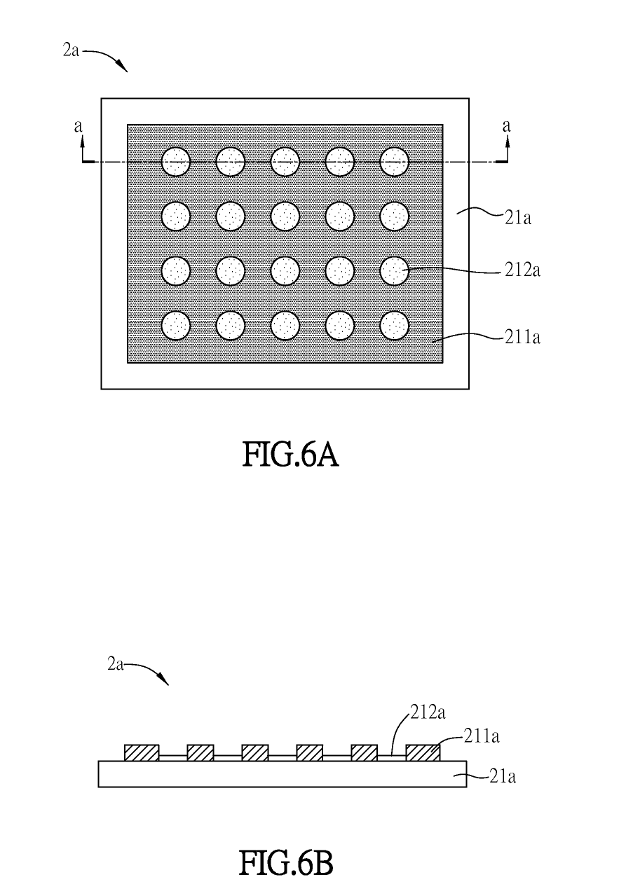

The above description is not for limiting the scope of the invention, and in other embodiments, the reaction zone also can be an additional fiber substrate disposed on the substrate. As shown in FIGS. 6A and 6B, like the foregoing embodiment, the reaction zone 212a of the detecting device 2a is defined by the non-reaction zone 211a formed by the wax printing method. However, the reaction zone 212a is formed by disposing another fiber substrate as the region for detecting the body fluid sample of the body surface. In this case, the substrate 21a is unnecessarily made by the high-density fiber substrate.

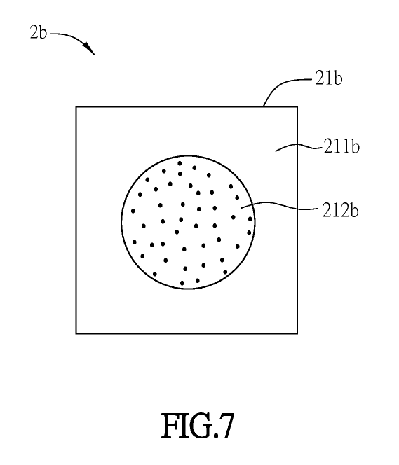

The number of the reaction zone is also not limited in this invention. In other embodiments, as shown in FIG. 7, the detecting substrate 21b of the detecting device 2b can have only one reaction zone 212b and one non-reaction zone 211b, and this can be designed according to the required condition during the detection.

As shown in FIGS. 1 and 5B, in this embodiment, the detecting device 2 is applied to the detection of bullous pemphigoid (BP). Since the BP patient can self-generate the antibody anti the COL17 antigen (or called BP180 and BPAG2), the detecting device 2 of this embodiment is used as the detecting reagent for the COL17 antigen C to execute the following ELISA. In this embodiment, a detecting reagent is immobilized in the fiber substrate within the reaction zone 212. In other words, the COL17 antigens C are immobilized to the fiber substrate. The number of the COL17 antigen C shown in FIG. 5B is just for the illustrative purpose but not for limiting the scope of the invention. The method of disposing the antibody to the detecting substrate can be comprehended by those skilled in the art, wherein, for example, the solution containing the COL17 antigens C is used to moisten the reaction zone 212 and then the moisture content is removed so as to achieve the immobilization, so the related description is omitted here for conciseness.

To be noted, in actual applications, the kind of the detecting reagent disposed in the reaction zone 212 can be changed according to the detection requirement but is not limited to the COL17 antigen C.

As shown in FIGS. 1A to 5B, when the detecting device 2 of the detection kit D is used to detect the BP and the body fluid sample collected by the sampling device 1 is disposed in the reaction zone 212, the operation is illustrated as below. When the collected body fluid sample contains the antibody (can be monoclonal or polyclonal antibody, but the monoclonal antibody more favorably) anti the COL17 antigen C, the COL17 antigen C in the reaction zone 212 will interact with the antibodies within the body fluid sample and especially with the antibodies which can form the specific binding with the NC16A domain (non-collagenous 16A domain) of the antigen. This invention is not limited to the ELISA detection method, and the operation thereof is illustrated as below. The antibody anti the COL17 antigen C can specifically identify the COL17 antigens C and specifically bind therewith. The extra and unbound body fluid of the body surface is eluted, and the secondary antibody carrying enzyme is joined to bond with the antibody anti COL17 antigen C. Then, the extra and un-bonded secondary antibody is eluted, and the enzyme substrate is joined to make the enzyme show color so that the patient can be judged if infected with BP. Therefore, the content of the antibody anti the COL17 antigen C can be estimated by the color of the colored product and the qualitative and quantitative purposes can be thus achieved at the same time.

To be noted, the method of judging the content of the antibody anti the COL17 antigen C includes, for example but is not limited to, coloring, fluorescent light, cold light, radiation or other types. In an embodiment, the ELISA method is to use the coloring enzyme and substrate showing the variable color to show the antigen or the subject. In other embodiments, the fluorescent light, cold light or real-time PCR can be used to generate the identifiable signals. The quantitative method is not limited in this invention, and the above-mentioned or related quantitative methods can be encompassed in the scope of the invention.

Subsequently, an experiment will be given to illustrate the detection kit and detection method of the invention with the operation and effect thereof and the details of the main steps of detecting the BP. To be noted, the following description is just for the illustrative purpose so that those skilled in the art can achieve the implementation accordingly, but not for limiting the scope of the invention.

Experiment 1: using a paper-based detecting device in the ELISA to detect the anti-NC16A antibody obtained from the body fluid of the body surface of the patient.

The steps include: providing a filter paper plate, moistening the filter paper plate and then joining the COL17 antigen having the NC16A domain (0.1 μg) in each of the reaction zones of the detecting device, adding the blister sample of the BP patient and the comparison blister sample which is obtained from the blister caused by the scald, pressure sore or frostbite, then adding the anti-IgG antibody connecting to HRP, after 20 minutes implementing PBST washing, and then adding the solution containing 3, 3′, 5, 5′-tetramethylbenzidine (TMB) and H₂O₂ for coloring.

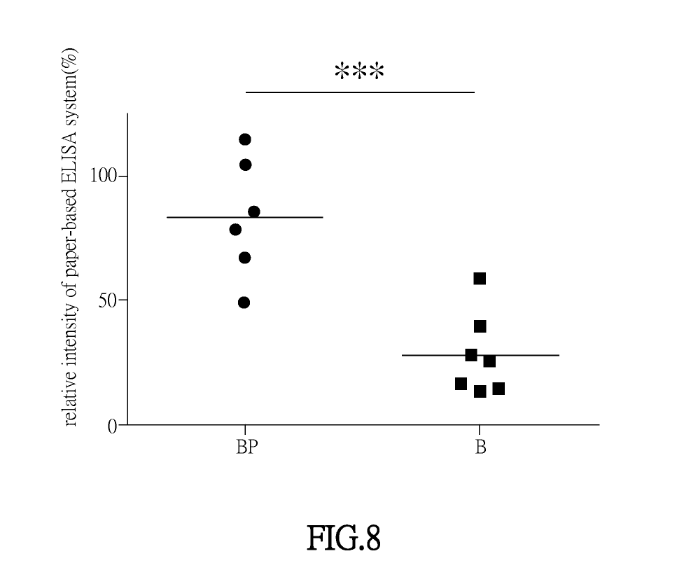

The detection result is shown in FIG. 8. The sample cell includes the body fluid of the body surface obtained from the BP patient (denoted by BP) and the body fluid of the body surface of the burned patient (denoted by B). The called body fluid of the body surface indicates the fluid of the blister of the BP patient and the fluid of the affected part of the burned patient. From FIG. 8, it can be known that although the detection samples of the two groups are both obtained from the body fluid of the affected part of the patient (especially the blister), the body fluid of the body surface of the BP patient has a higher content of the anti-NC16A antibody and the distribution of the relative intensity thereof is obviously higher than the case of the body fluid of the burned patient (B), demonstrating that the anti-NC16A antibody of the affected part of the BP patient undoubtedly can be used as the detection target of the BP detection.

Summarily, in the detection kit and detection method of the invention, the sampling device and the detecting device can effectively implement the collection and detection to the body fluid sample. The sampling unit of the sampling device is covered with a filtering unit, which can effectively separate non detection target from the body fluid sample so that the sampling unit can effectively collect the desired target from the body fluid sample. Thereby, the pretreatment and the pretreatment time for the body fluid sample can be lessened and the problem of losing body fluid sample due to the extra processing steps can be avoided. Therefore, the required amount of the sample can be reduced. Besides, the detection kit of the invention includes a detecting device, so the detection purpose can be achieved by making the body fluid sample that is filtered and absorbed by the sampling unit contact the detecting device.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A detection kit applied for detecting a body fluid sample of an organism, comprising:
a sampling device including:
a sampling unit; and
a filtering unit detachably covering the sampling unit, wherein the sampling unit collects the body fluid sample that is filtered by the filtering unit; and
a detecting device including at least one reaction zone including a fiber substrate and a detecting reagent immobilized to the fiber substrate,
wherein the reaction zone is disposed as the fiber substrate contacts the body fluid sample collected by the sampling unit, and the fiber substrate absorbs the body fluid sample collected by the sampling unit, and the body fluid sample reacts with the detecting reagent for the detection.

2. The detection kit as recited in claim 1, wherein the detecting device includes at least one non-reaction zone which is covered with hydrophobic material.

3. The detection kit as recited in claim 2, wherein the non-reaction zone surrounds the reaction zone to expose the fiber substrate to absorb the body fluid sample collected by the sampling unit.

4. The detection kit as recited in claim 1, which is the detection kit for the bullous pemphigoid (BP).

5. The detection kit as recited in claim 4, wherein the detecting reagent includes a plurality of COL17 antigens.

6. The detection kit as recited in claim 1, wherein the material of the filtering unit includes gauze, nylon cloth, non-woven cloth or their any combination.

7. A detection method for detecting a body fluid sample of an organism, comprising steps of:
providing a sampling device and a detecting device, wherein the sampling device includes a sampling unit and a filtering unit detachably covering the sampling unit, the detecting device has at least one reaction zone which includes a fiber substrate and a detecting reagent immobilized to the fiber substrate;
making the sampling unit covered by the filtering unit contact the body fluid sample so that the body fluid sample is absorbed by the sampling device;
separating the filtering unit from the sampling unit and making the body fluid sample absorbed by the sampling unit contact the reaction zone; and
detecting the body fluid sample.

8. The detection method as recited in claim 7, which is the detection method for the BP.

9. The detection method as recited in claim 8, wherein the detecting device includes a plurality of COL17 antigens immobilized to the fiber substrate.

10. The detection method as recited in claim 9, further comprising a step of:
detecting the interaction between the body fluid sample and the COL17 antigens.
